(19) United States
(12) Patent Application Publication
(10) Pub. No.: US 2023/0172542 A1
(43) Pub. Date: Jun. 8, 2023

(54) System, Method and Apparatus for Control of Shivering During Targeted Temperature Management

(71) Applicant: C. R. BARD, INC., New Jersey, NJ (US)

(72) Inventors: Jeff Baker, Durham, NC (US); Patrick Downie, Apex, NC (US); Jesse Smith, Broomfield, CO (US); Eric A. Fallows, Apex, NC (US); Michael Yarger, Chapel Hill, NC (US); David Bostick, Wilmington, DE (US)

(21) Appl. No.: 17/923,813

(22) PCT Filed: May 11, 2021
(86) PCT No.: PCT/US21/31837

§ 371 Date: Nov. 7, 2022

Related U.S. Application Data
(60) Provisional application No. 63/023,633, filed on May 12, 2020.

(51) Int. Cl. A61B 5/00

(52) U.S. Cl. CPC A61B 5/6803; A61B 5/7264; A61B 5/291

(57) ABSTRACT

Disclosed herein is a system, apparatus and method directed to patient temperature control. The system can include a control module configured to provide fluid that is heated or cooled, a heating and cooling system configured to couple with the control module and receive the fluid, and a temperature sensor coupled to the control module, the temperature sensor configured to measure a body temperature of a patient and provide signals to the control module that indicate the body temperature. The system can include a neurostimulation device coupled to the control module to provide neurostimulation to the patient. The control module can include logic, stored on non-transitory, computer-readable medium that, when executed by one or more processors, causes performance of operations including generation and transmission of first instructions to the neurostimulation device causing initiation of a first neurostimulation procedure.

### PRIORITY

This application claims the benefit of priority to U.S. Provisional Application No. 63/023,633, filed May 12, 2020, which is incorporated by reference in its entirety into this application.

### SUMMARY

Briefly summarized, embodiments disclosed herein are directed to systems, methods and apparatuses for controlling shivering of a patient undergoing targeted temperature management through the use of neurostimulation techniques.

Some embodiments of the disclosure describe the use of neurostimulation to control (e.g., reduce or prevent) patient shiver during targeted temperature management (TTM), or otherwise known as therapeutic hypothermia. As one known use, TTM is a neuroprotectant therapy that lowers or maintains a patient's body temperature during recovery typically following a period of stopped or reduced blood flow to the brain (e.g. such as from cardiac arrest or stroke). Such an application of TTM therapy is believed to lower the risk of brain injury by slowing destructive metabolic pathways after the return of spontaneous blood circulation.

There are certain challenges that arise when providing TTM therapy to a patient. For instance, one common challenge clinicians face with providing TTM therapy is reducing patient shiver during TTM. Specifically, shiver causes an increase in metabolic rate and an increase in body temperature, which increases the difficulty in providing precise TTM therapy. In order to solve the shiver problem today, clinicians may increase the target temperature of the therapy. Alternatively, or in addition, clinicians may administer paralytic or sedative drugs to reduce shiver during TTM therapy. Yet another current solution to shiver includes increasing the core temperature, which adversely affects the TTM therapy. Further, in the current solutions, first clinicians need to identify shiver in patients undergoing hypothermia, which is a common technical challenge.

The disclosure describes various embodiments that utilize neurostimulation to control shiver prior to, during, or after the application of TTM therapy. Neurostimulation, or neuromodulation, may be defined as intentional modulation of a patient's nervous system's activity using invasive or non-invasive means. One type of neurostimulation, deep brain stimulation (DBS), has been used since approximately 1997 to reduce tremor in patients with movement disorders such as Parkinson's disease. Specifically, DBS is an invasive neurosurgical procedure involving insertion of a neurostimulator in the brain. The neurostimulator transmits electrical impulses through implanted electrodes to specific targets in the brain. Although DBS is an invasive method, non-invasive approaches such as transcranial magnetic stimulation or direct current stimulation, are possible alternatives and may be used interchangeably to provide neurostimulation as discussed below, unless explicitly noted or the embodiment inherently excludes one or more methods.

Instead of utilizing DBS to target the thalamus (i.e., as in the treatment of Parkinson's disease), embodiments of the disclosure are directed to utilizing DBS, or other neurostimulation methods, to target the hypothalamus, which is an area of the brain in close proximity to the thalamus. As referenced above, the hypothalamus is the region of the brain that controls thermoregulation and shiver.

Various embodiments disclosed as any of a system, a method or an apparatus are directed to stimulating the hypothalamus of a patient, which is the area of the brain that controls thermoregulation and shiver. One embodiment includes a system comprising a targeted temperature system in combination with a neurostimulation device (referred to herein as a TTM/neurostimulation system). The targeted temperature management system may include any number of systems that provide TTM therapy, including for example, any of the systems or portions thereof described in, at least, U.S. Pat. No. 6,645,232, hereby incorporated by reference in its entirety.

As referenced above, either invasive or non-invasive neurostimulation methods may be utilized. Various apparatuses or components may be included within the system to provide the neurostimulation as will be discussed below. Further, the TTM/neurostimulation system may include one or more sensors or monitoring devices configured to monitor the shivering of a patient undergoing TTM therapy. In various embodiments of the disclosure, logic controlling the neurostimulation may be stored on the neurostimulation device, the targeted temperature system, and/or a network device such as a mobile device or a server device.

It should be noted that embodiments of the disclosure may reduce or even eliminate the need to detect shiver as, at least in some embodiments, shiver may be prevented.

In one embodiment, a patient temperature control system is disclosed that comprises a control module configured to provide fluid that is heated or cooled, wherein the control module includes a circulating pump, a heating and cooling system configured to couple with the control module and receive the fluid, wherein circulating pump is configured to circulating the fluid through the heating and cooling system, a temperature sensor coupled to the control module, the temperature sensor configured to measure a body temperature of a patient and provide signals to the control module that indicate the body temperature, and a neurostimulation device coupled to the control module and configured to provide neurostimulation to the patient, wherein the control module includes logic, stored on non-transitory, computer-readable medium that, when executed by one or more processors, causes performance of operations including generation and transmission of first instructions to the neurostimulation device causing initiation of a first neurostimulation procedure.

In some embodiments, the control module includes a heat exchange system for heating or cooling the fluid. In some embodiments, the logic, when executed by the one or more processors, causes performance of further operations including receiving the signals indicating the body temperature, analyzing the body temperature to determine whether a temperature of the fluid is to be adjusted, and providing second instructions to the heat exchange system indicating whether the fluid is to be heated, cooled or maintained at a current temperature.

In yet other embodiments, the control module further includes a fluid reservoir that is maintained at substantially atmospheric pressure. In some embodiments, patient temperature control system further comprises a motion sensor device configured to measure an amount of motion of the patient indicating a level of shiver. In some embodiments, the heating and cooling system includes one or more contact pads configured to directly or indirect contact the patient and receive the fluid. In some embodiments, the neurostimulation device includes an external electrode and an internal electrode, and is configured to provide deep brain stimulation. In other embodiments, the neurostimulation device includes an external electrode and an internal electrode placed within the patient's skull, and is configured to provide deep brain stimulation. In yet other embodiments, the neurostimulation device includes at least a first set of electrodes for placement on the patient's skull, the first set of electrodes including an emitter and a receiver each configured to receive power via a power supply device, wherein the neurostimulation device is configured to provide noninvasive brain stimulation. Still, in other embodiments, the neurostimulation device is a wrist-worn device. In some embodiments, the neurostimulation is configured to target the hypothalamus to control thermoregulation of the patient's body and prevent or reduce patient shiver.

Embodiments of the disclosure disclose a method of providing targeted temperature management and neurostimulation, wherein the method comprises operations of providing a targeted temperature management (TTM) procedure to a patient by circulating cooled fluid through a heating and cooling system directly or indirectly in contact with the patient, obtaining a measurement indicating an amount of shiver by a body of the patient, determining, based at least in part on the measurement indicating the amount of shiver, a neurostimulation procedure to be initiated on the patient, and providing the neurostimulation procedure to the patient. In some embodiments, the TTM procedure is provided by a combination of a control module configured to provide the cooled fluid and a circulating pump configured to circulate the cooled fluid through the heating and cooling system.

In other embodiments, the control module receives a measurement of a body temperature of the patient from a temperature sensor. In some embodiments, the control module includes logic, stored on non-transitory, computer-readable medium that, when executed by one or more processors, causes performance of operations including generation and transmission of first instructions to a neurostimulation device causing the providing of the neurostimulation procedure to the patient.

In other embodiments, the neurostimulation device includes an external electrode and an internal electrode placed within the patient's skull, and is configured to provide deep brain stimulation. In yet other embodiments, the neurostimulation device includes at least a first set of electrodes for placement on the patient's skull, the first set of electrodes including an emitter and a receiver each configured to receive power via a power supply device, wherein the neurostimulation device is configured to provide noninvasive brain stimulation. Still in other embodiments, the neurostimulation device is a wrist-worn device. In some embodiments, the neurostimulation procedure is configured to target the hypothalamus to control thermoregulation of the patient's body and prevent or reduce patient shiver. In yet other embodiments, the control module further includes a fluid reservoir that is maintained at substantially atmospheric pressure.

These and other features of the concepts provided herein will become more apparent to those of skill in the art in view of the accompanying drawings and following description, which disclose particular embodiments of such concepts in greater detail.

### BRIEF DESCRIPTION OF THE DRAWINGS

Embodiments of the disclosure are illustrated by way of example and not by way of limitation in the figures of the accompanying drawings, in which like references indicate similar elements and in which:

FIG. 1 illustrates a block diagram of a targeted temperature management (TTM)/neurostimulation system according to some embodiments;

FIG. 2 illustrates a block diagram of the control module of the TTM/neurostimulation system of FIG. 1 according to some embodiments;

FIG. 3 illustrates a flowchart illustrating an exemplary method of providing TTM therapy with the TTM/neurostimulation system of FIG. 1 according to some embodiments;

FIGS. 4A-4F illustrate example neurostimulation devices of the TTM/neurostimulation system of FIG. 1 according to some embodiments;

FIGS. 5A-5D illustrate example monitoring devices of the TTM/neurostimulation system of FIG. 1 according to some embodiments;

FIG. 6 is a logical representation of the control module of FIG. 2 according to some embodiments;

FIGS. 7A-7B illustrate example user interface displays employable in conjunction with implementations of the TTM/neurostimulation system of FIG. 1 according to some embodiments;

FIG. 8 is a logical representation of logic configured to control aspects of the TTM/neurostimulation system of FIG. 1 according to some embodiments.

### DETAILED DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal end portion” of, for example, a probe disclosed herein includes a portion of the probe intended to be near a clinician when the probe is used on a patient. Likewise, a “proximal length” of, for example, the probe includes a length of the probe intended to be near the clinician when the probe is used on the patient. A “proximal end” of, for example, the probe includes an end of the probe intended to be near the clinician when the probe is used on the patient. The proximal portion, the proximal end portion, or the proximal length of the probe can include the proximal end of the probe; however, the proximal portion, the proximal end portion, or the proximal length of the probe need not include the proximal end of the probe. That is, unless context suggests otherwise, the proximal portion, the proximal end portion, or the proximal length of the probe is not a terminal portion or terminal length of the probe.

With respect to “distal,” a “distal portion” or a “distal end portion” of, for example, a probe disclosed herein includes a portion of the probe intended to be near or in a patient when the probe is used on the patient. Likewise, a “distal length” of, for example, the probe includes a length of the probe intended to be near or in the patient when the probe is used on the patient. A “distal end” of, for example, the probe includes an end of the probe intended to be near or in the patient when the probe is used on the patient. The distal portion, the distal end portion, or the distal length of the probe can include the distal end of the probe; however, the distal portion, the distal end portion, or the distal length of the probe need not include the distal end of the probe. That is, unless context suggests otherwise, the distal portion, the distal end portion, or the distal length of the probe is not a terminal portion or terminal length of the probe.

The term “logic” may be representative of hardware, firmware or software that is configured to perform one or more functions. As hardware, the term logic may refer to or include circuitry having data processing and/or storage functionality. Examples of such circuitry may include, but are not limited or restricted to a hardware processor (e.g., microprocessor, one or more processor cores, a digital signal processor, a programmable gate array, a microcontroller, an application specific integrated circuit “ASIC”, etc.), a semiconductor memory, or combinatorial elements.

Additionally, or in the alternative, the term logic may refer to or include software such as one or more processes, one or more instances, Application Programming Interface(s) (API), subroutine(s), function(s), applet(s), servlet(s), routine(s), source code, object code, shared library/dynamic link library (dll), or even one or more instructions. This software may be stored in any type of a suitable non-transitory storage medium, or transitory storage medium (e.g., electrical, optical, acoustical or other form of propagated signals such as carrier waves, infrared signals, or digital signals). Examples of a non-transitory storage medium may include, but are not limited or restricted to a programmable circuit; non-persistent storage such as volatile memory (e.g., any type of random access memory “RAM”); or persistent storage such as non-volatile memory (e.g., read-only memory “ROM”, power-backed RAM, flash memory, phase-change memory, etc.), a solid-state drive, hard disk drive, an optical disc drive, or a portable memory device. As firmware, the logic may be stored in persistent storage.

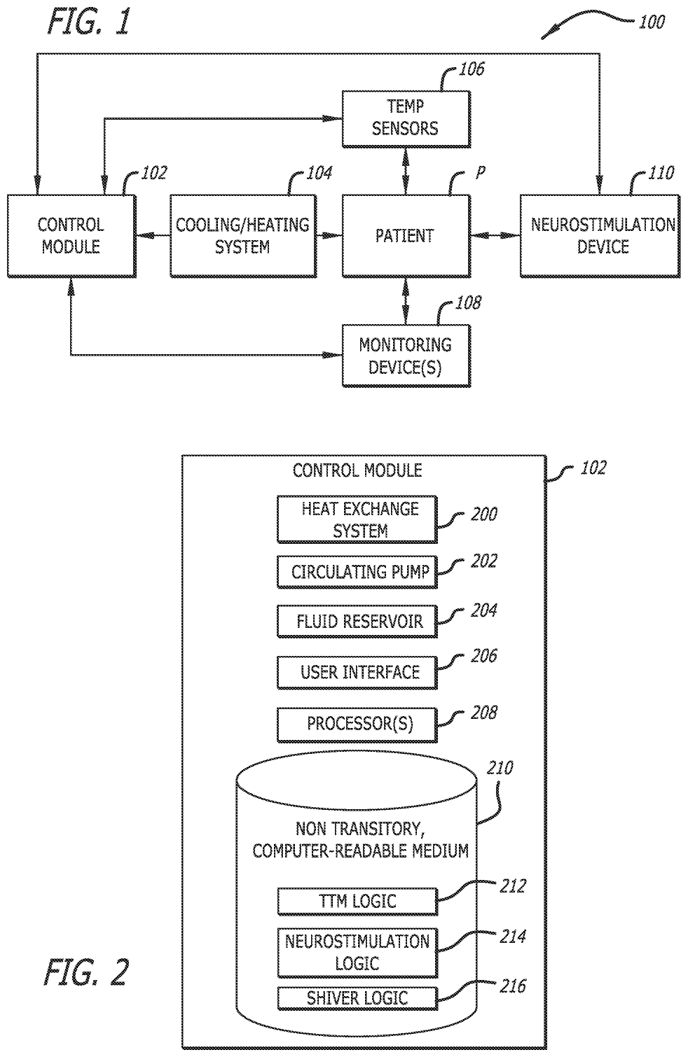

Referring now to FIG. 1, a block diagram of a targeted temperature management (TTM)/neurostimulation system is shown according to some embodiments. As shown, a targeted temperature management (TTM) and neurostimulation system (“TTM/neurostimulation system” or “system”) 100 includes a control module, a cooling/heating system 104, temperature sensor(s) 106, monitoring device(s) 108 and a neurostimulation device 110. The system 100 is shown as being configured to monitor at least one physiological response of a patient P based on a change of temperature of the patient resulting from application of TTM therapy.

For instance, the TTM therapy may include therapeutic hypothermia that is induced by the cooling/heating system 104. The cooling/heating system 104 may comprise any of a number of different modalities for selective cooling of a patient, including for example cooled contact pads, vascular cooling, patient emersion approaches and/or other systems for rapidly cooling a patient, e.g. systems as described in U.S. Pat. Nos. 6,669,715; 6,827,728; 6,375,674; 6,645,232, and PCT publication No. WO/2007/121480, each of which is incorporated by reference in its entirety into this application.

As will be discussed in further detail below, the control module 102 includes logic and processor(s)/circuitry that, upon execution and processing, may be configured to analyze signals provided by any of the temperature sensors 106, the monitoring device 108 (“monitoring signals”), and the neurostimulation device 110. For example, the temperature sensors 106 may provide the control module 102 with signals indicating a temperature of the patient P. The control module 102 may in turn alter, or maintain, the cooling/heating efforts of the cooling/heating system 104. In one embodiment, the cooling/heating efforts may refer to the temperature of fluid circulating from the control module 102 through the cooling/heating system 104 (e.g., contact pads or a catheter). Herein, the term “pads” will be used to refer to the cooling/heating system 104; however, such is not intended to be limiting of other possible embodiments of the cooling/heating system 104.

In addition to the functionalities described herein, the processors/circuitry of the control module 102 may be further adapted for providing instructions, via one or more input signals, to the pads 104. Such instructions may be based on signals received from the temperature sensors and an established degree of cooling and/or rate of cooling of the given patient P. For example, based upon a measured patient temperature the cooling/heating rate may be adjusted (or maintained). Additionally, as will be discussed below, signals from the monitoring device 108 may be utilized by the control module 102 to generate instructions provided to the neurostimulation device 110, via one or more input signals, to initiate a stimulation procedure, or alter or maintain a current stimulation procedure.

Additionally, the monitoring signals may indicate a shivering response of the patient P resulting, at least in part, from application of the TTM therapy. In one embodiment, the shivering response may be utilized to provide a visual and/or audible output via a user interface of the control module 102 or other output device (e.g. one or more light emitting diodes (LEDs)) and/or a network device communicatively coupled to the control module 102. In some embodiments, the output provides an indication of a magnitude, degree and/or stage of a patient shivering response to the TTM therapy.

As may be appreciated, the processors/circuitry and a user interface of the control module 102 may be provided for interactive operations therebetween. More particularly, in conjunction with a given patient cooling procedure, a user may utilize the user interface to access and select a given one of a plurality of treatment protocols, e.g. corresponding with a given protocol established at a given user site (e.g. for a particular physician). In turn, such protocol may provide for the selection of a given neurostimulation treatment (e.g. via an interactive menu).

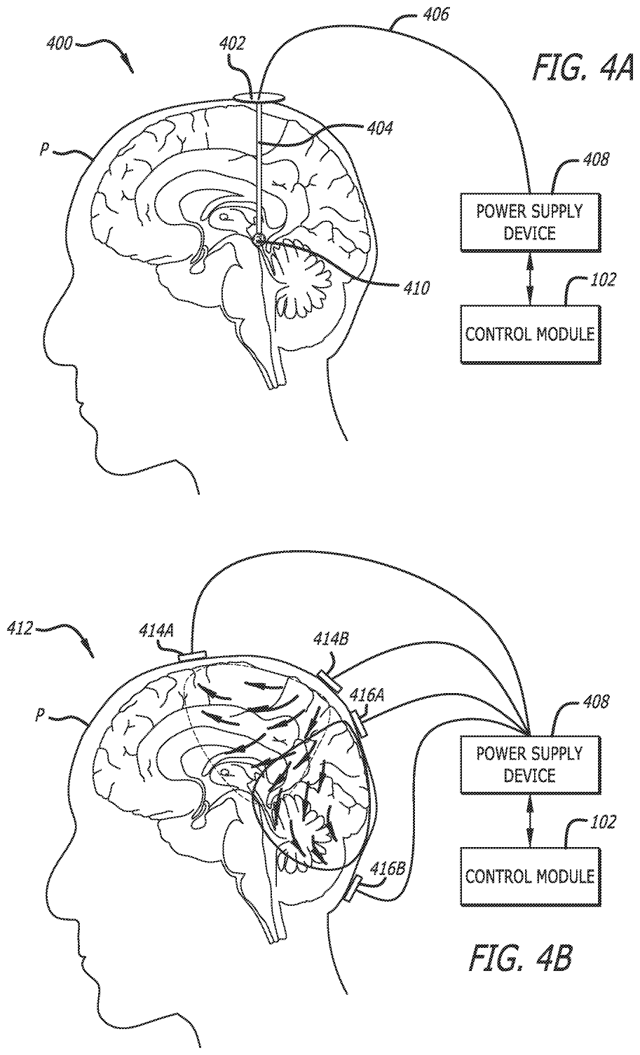

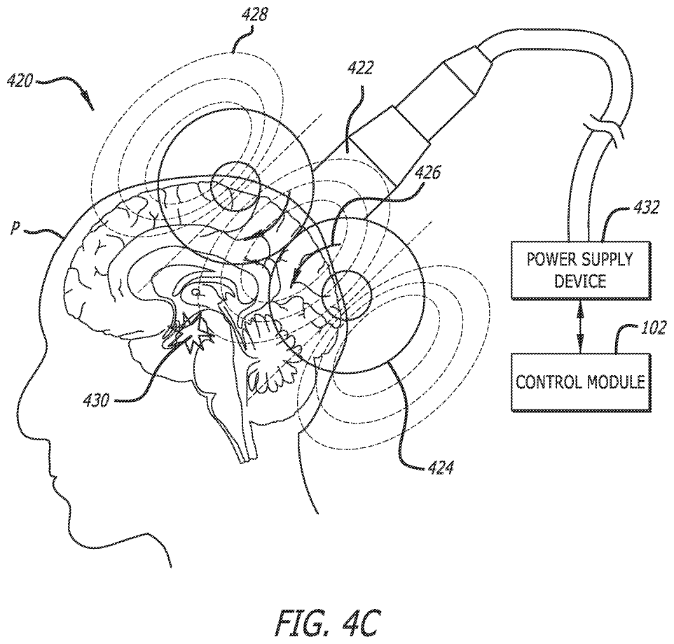

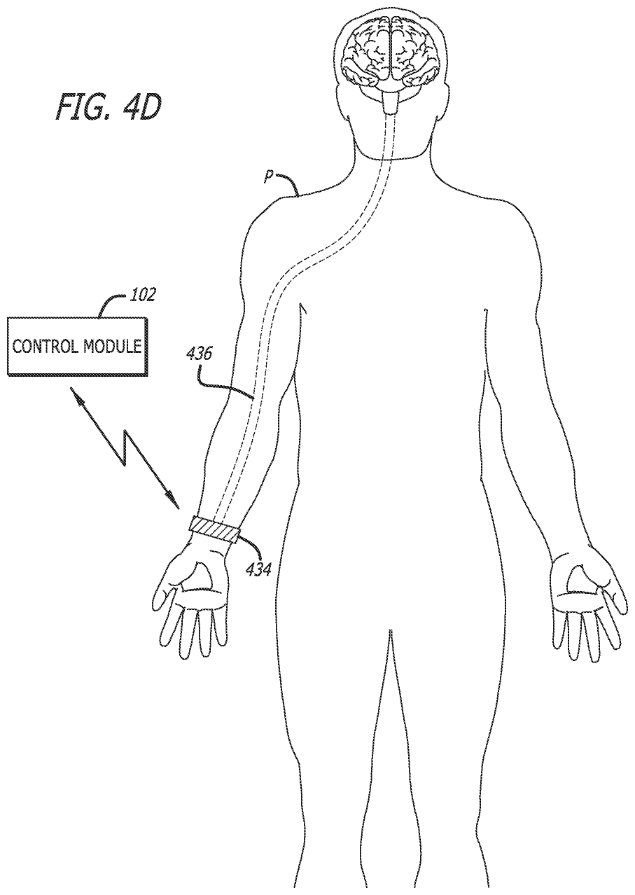

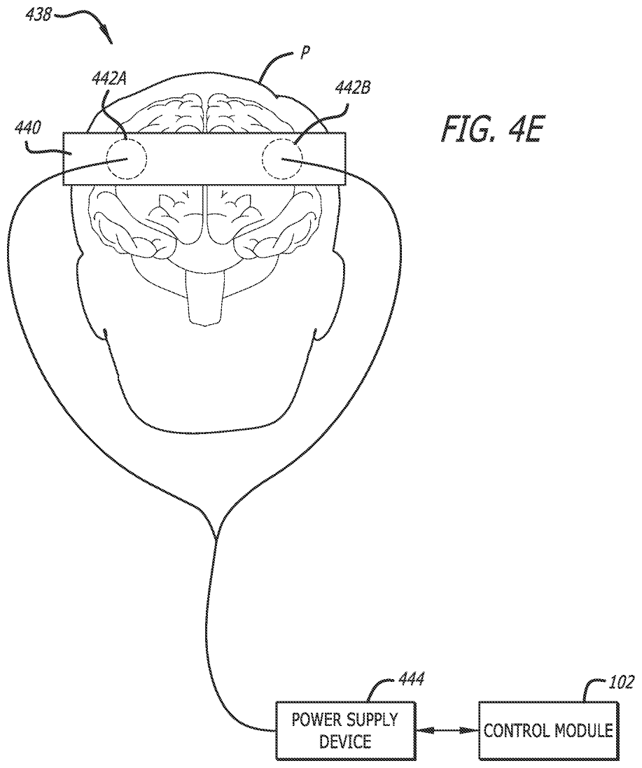

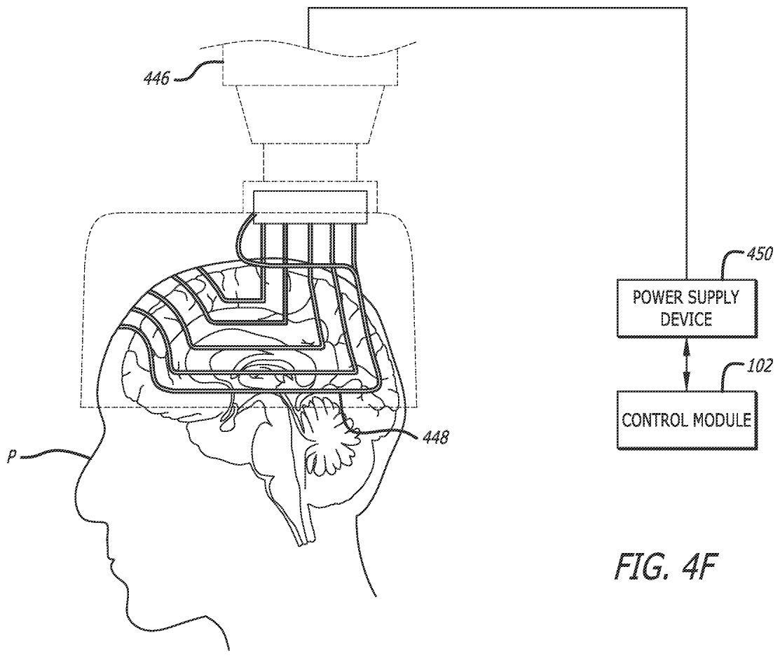

In turn, for a selected neurostimulation treatment, the processors/circuitry may be operative to provide instructions via a wired or wireless coupling to the neurostimulation device 110. Such information may be provided so as to take into account specific data inputted by a user at the user interface for a given TTM therapy procedure, including for example, patient-specific information (e.g. age, weight, gender, etc.), and patient procedure-specific information (e.g. thermotherapy pursuant to stroke, thermotherapy pursuant to head trauma, etc.). Additionally, and/or alternatively, the information comprising the output may be based, at least in part, upon a magnitude of the measured patient response reflected by the monitoring signals. For example, a magnitude measure may be obtained from one or more monitoring signals and compared with pre-established reference data to determine the applicable duration, type and/or intensity of stimulation provided by the neurostimulation device 110. Examples of the neurostimulation device 110 include, but are not limited or restricted to, those illustrated in FIGS. 4A-4F.

Additionally, the processors/circuitry of the control module 102 may further comprise and/or execute logic comprising algorithms and/or data for processing the signals received from any of the temperature sensors 106, the monitoring device 108, and the neurostimulation device 110 as well as user input received via the user interface. Receipt of the signals and/or user input may be on an ongoing basis, e.g. after initiation TTM therapy via the pads 104 and/or the neurostimulation procedure via the neurostimulation device 110, to assess the effectiveness of such procedures, wherein such assessment may then be automatically employed in the generation of subsequent instructions to either of the cooling/heating system 104 and/or the neurostimulation device 110, which may alter or maintain either procedure.

The signals and the user input may be stored in non-transitory computer-readable medium of the control module 102, or otherwise accessible thereby. By way of example, the above-noted analyses and determinations may include an algorithmic analysis as to the degree of patient shivering reduction, the duration of shivering reduction and/or the degree of shivering reduction on a time-scale basis associated with a given neurostimulation procedure (e.g. collectively “trend data”). Ongoing treatment information and/or future anticipated treatment information as determined through such analyses or determinations may be provided to a user through the user interface, wherein such further information is based in part on the trend data assessment.

Referring now to FIG. 2, a block diagram of the control module of the TTM/neurostimulation system of FIG. 1 is shown according to some embodiments. The control module 102 is seen to include a plurality of components including at least a heat exchange system 200, a circulating pump 202, a fluid reservoir 204, a user interface 206, one or more processors (“processor”) 208, and non-transitory computer-readable medium 210 that has stored thereon TTM logic 212, neurostimulation logic 214, and shiver logic 216.

The heat exchange system 200 is considered for affecting at least one of heating or cooling a fluid that circulates through the pads 104. The circulating pump 202 is configured to circulate the fluid through the heat exchange system 200 and the pads 104 to affect heat transfer between the pads 104 and the patient P.

The control module 102 may also include a fluid reservoir 204 that is fluidly interconnectable with the pads 104. The fluid reservoir 204 may be utilized to contain fluid that is removable from the reservoir to fill/circulate through the pads 104 during use. In conjunction with this aspect, the system may be defined so that, during normal heating/cooling operations, fluid is circulatable through the pads 204 and the heat exchange system 200 by the circulating pump 202. Additional embodiments have been contemplated in which the control module 102 includes a plurality of fluid reservoirs as described in U.S. Pat. No. 6,645,232, which is incorporated by reference in its entirety into this application.

Additionally, the control module 102 may include the user interface 206 as an output device for providing output information in at least one of an audible and visual form as described here. By way of example, the information may be provided via an interactive display (e.g., a touchscreen) and, optionally, physical input buttons.

The control module 102 may include the non-transitory computer-readable medium 210 that includes several logic modules, the operations of which are described herein. Generally, the TTM logic 212 includes logic, algorithms and/or data that, upon execution by the processor 208, cause performance of operations that instruct the heat exchange system 200 to warm or cool fluid circulating through the pads 104. In generating instructions for the heat exchange system 200, the TTM logic 212 may consider several parameters including, one or more of, profile information of the patient P (e.g., age, gender, weight, etc.), the number of pads 104 (and the extent to which the pads 104 cover the patient P), the signals received from the temperature sensors 106 and the monitoring device 108, and any user input.

Similarly, the neurostimulation logic 214 includes logic, algorithms and/or data that, upon execution by the processor 208, cause performance of operations that instruct the neurostimulation device 110 to initiate, alter or terminate a neurostimulation procedure on the patient P. In generating instructions for the neurostimulation device 110, the neurostimulation logic 214 may consider several parameters including, one or more of, profile information of the patient P, the neurostimulation device 110 (as the system 100 is configured to utilize several embodiments of neurostimulation devices), the signals received from the temperature sensors 106 and the monitoring device 108, and any user input.

Additionally, the shiver logic 216 includes logic, algorithms and/or data that, upon execution by the processor 208, cause performance of operations that analyze the signals received from at least the monitoring device 108 to determine a level of shiver of the patient P. Such determination may be provided to the TTM logic 212 and/or the neurostimulation logic 214. It should be noted that any of the determinations, assessments or analyses of the logic discussed herein may be provided or indicated via alerts, alarms, or general displays via the user interface 206 or otherwise (e.g., other displays, speakers, or via communication of such to remote displays or speakers, such as of a network device, e.g., a mobile device, communicatively coupled to the control module 102).

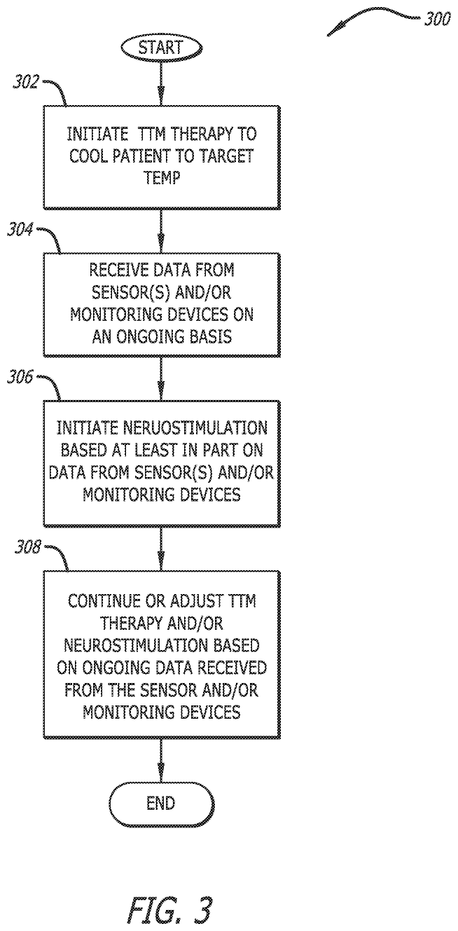

Referring to FIG. 3, a flowchart illustrating an exemplary method of providing TTM therapy in conjunction with a neurostimulation procedure with the TTM/neurostimulation system of FIG. 1 is shown according to some embodiments. Each block illustrated in FIG. 3 represents an operation performed in the method 300 of providing TTM therapy and a neurostimulation procedure using the TTM/neurostimulation system 100. Herein, the method 300 starts with the initiation of a TTM therapy directed at cooling a patient (block 302). Following initiation of the TTM therapy, the system 100 receives data (signals) from the temperature sensors 106 and/or the monitoring device 108 on an ongoing basis (e.g., such as at periodic or aperiodic intervals) (block 304).

Based on the signals received and on determinations or analyses performed by one or more of the TTM logic 212, the neurostimulation logic 214 and/or the shiver logic 216, the neurostimulation logic 214 causes initiation of a neurostimulation procedure (block 306). The initiation of the neurostimulation procedure may be via instructions provided to a neurostimulation device either via wired or wireless communication.

The method 300 continues the TTM therapy and/or the neurostimulation procedure based on analysis of the ongoing reception of the signals from the temperature sensors 106 and/or the monitoring device 108 by one or more of the TTM logic 212, the neurostimulation logic 214 and/or the shiver logic 216 (block 308). For example, the intensity of the neurostimulation procedure may be reduced following a period when a magnitude of shiver of the patient P is below a set threshold. In other instances, the intensity of the neurostimulation procedure may be increased following a period when the magnitude of shiver of the patient P is after a set threshold with the expectation that an increase in intensity will reduced or eliminate the shiver of the patient P. It should be understood that as the analyses performed at block 308 may be performed at intervals throughout the entire duration of the TTM therapy in order to maintain a predetermined level of shiver (or eliminate/prevent shiver altogether).

Referring to FIGS. 4A-4F, example neurostimulation devices of the TTM/neurostimulation system of FIG. 1 are shown according to some embodiments. The devices illustrated in FIGS. 4A-4F are merely examples and are not intended to limit the disclosure. Instead, the embodiments illustrated provide one of ordinary skill in the art numerous examples are possible neurostimulation devices that may be included within the system 100. Referring now to FIG. 4A, a neurostimulation device 400 for providing deep brain stimulation (DBS) is shown to include an external electrode 402, an internal electrode 404, a wire 406. The neurostimulation device 400 is connected to power supply device 408 via the wire 406 and to the control module 102, via wired or wireless coupling. As shown, the internal electrode 406 is placed within the brain, e.g., via surgical implantation. The neurostimulation device 400 operates to provide electric stimulation to certain areas of the brain (e.g., 410), such as the hypothalamus to control or regulate thermoregulation of the body of the patient P. Specifically, the electric stimulation comprises intentional modulation of the nervous system of the patient P, and based on the electric stimulation (such as the rate at which electric signals are provided), cause the patient P's body to cease shivering (or prevent shiver).

Referring to FIG. 4B, a neurostimulation device 412 for providing noninvasive DBS is shown to include two sets of electrodes each including an emitter and a receiver (e.g., emitters 414A, 416A and receivers 414B, 416B) and a wire extending from each electrode to a power supply device 418. The neurostimulation device 412 is connected to the control module 102, via wired or wireless coupling. In contrast to the neurostimulation device 400 of FIG. 4A, the neurostimulation device 412 provides noninvasive DBS via electric stimulation to certain areas of the brain, such as the hypothalamus. The neurostimulation device 412 causes electrical signals to be transmitted from a receiver to an emitter (e.g., emitter 414A to receiver 414B) at a predetermined frequency to cause intentional modulation of the nervous system of the patient P, and based on the electric stimulation (such as the rate at which electric signals are provided), cause the patient P's body to cease shivering (or prevent shiver). In some embodiments, such as the one shown in FIG. 4B, the electric stimulation of each set of electrodes may be provided specifically to interfere with each other, causing the electric signals to reach certain areas of the brain (e.g., the hypothalamus), that would otherwise not be reachable without the interference as the interference may redirect the electrical signals from the intended receiver.

Referring now to FIG. 4C, a neurostimulation device 420 for providing transcranial magnetic stimulation (TMS) is shown to include a handle 422 and TMS coils 424. The neurostimulation device 420 is connected to the power supply device 432 and to the control module 102, via wired or wireless coupling. The neurostimulation device 420 provides noninvasive TMS by supplying electric current 426 to the TMS coils 424, which causes the generation of the magnetic fields 428. The magnetic fields 428 cause the generation of electric current 430 within the brain of patient P. The electric current 430 within the brain causes intentional modulation of the nervous system of the patient P, which causes the patient P's body to cease shivering (or prevent shiver) by targeting the hypothalamus. Specific placement of the neurostimulation device 420 near the patient P's head may cause the electric current 430 to be generated within the brain specifically targeting the hypothalamus, for example.

Referring to FIG. 4D, a neurostimulation device 434 for providing neuromodulation therapy is shown as a wrist-worn device. The neurostimulation device 434 may include a battery, not shown, and may be wirelessly connected to the control module 102. However, in some embodiments, the neurostimulation device 434 may be configured to couple to the control module 102 via a wired connection. The neurostimulation device 434 provides neuromodulation therapy targeting certain areas of the brain, such as the hypothalamus, by issuing electrical stimulation to nerves via contact between the device 434 and skin of the patient P. The electrical stimulation causes electrical signals 436 to reach targeted areas of the brain causing intentional modulation of the nervous system of the patient P. The intentional modulation of the nervous system has the effect of causing the patient P's body to cease shivering (or prevent shiver) by targeting the hypothalamus.

Referring to FIG. 4E, a neurostimulation device 440 for providing transcranial direct current stimulation (tDCS) is shown to include a plurality of electrodes 442A-442B incorporated into a headband configured to be placed around the head of patient P. In the illustration, two electrodes are included within the device 440 but the device may include an alternative number of electrodes. Additionally, FIG. 4E illustrates a wire extending from each electrode 442A-442B to a power supply device 444. In alternative embodiments, the power supply device 444 may be a battery that is incorporated into the headband for example. The neurostimulation device 440 is connected to the control module 102, via wired or wireless coupling. The device 440 utilizes tDCS to cause intentional modulation of the nervous system of the patient P through transcranial magnetic stimulation (TMS) thereby causing the patient P's body to cease shivering (or prevent shiver).

Referring to FIG. 4F, a neurostimulation device 446 for providing deep transcranial magnetic stimulation (dTMS) is shown to include TMS coil elements 448. The neurostimulation device 446 is connected to the power supply device 450 and to the control module 102, via wired or wireless coupling. The neurostimulation device 446 provides noninvasive dTMS through the generation of electromagnetic fields by supply electric current to the TMS coil elements 448. The magnetic field(s) cause the generation of electric current within the brain of patient P, which in turn causes intentional modulation of the nervous system of the patient P. The intentional modulation of the nervous system has the effect of causing the patient P's body to cease shivering (or prevent shiver) by targeting the hypothalamus.

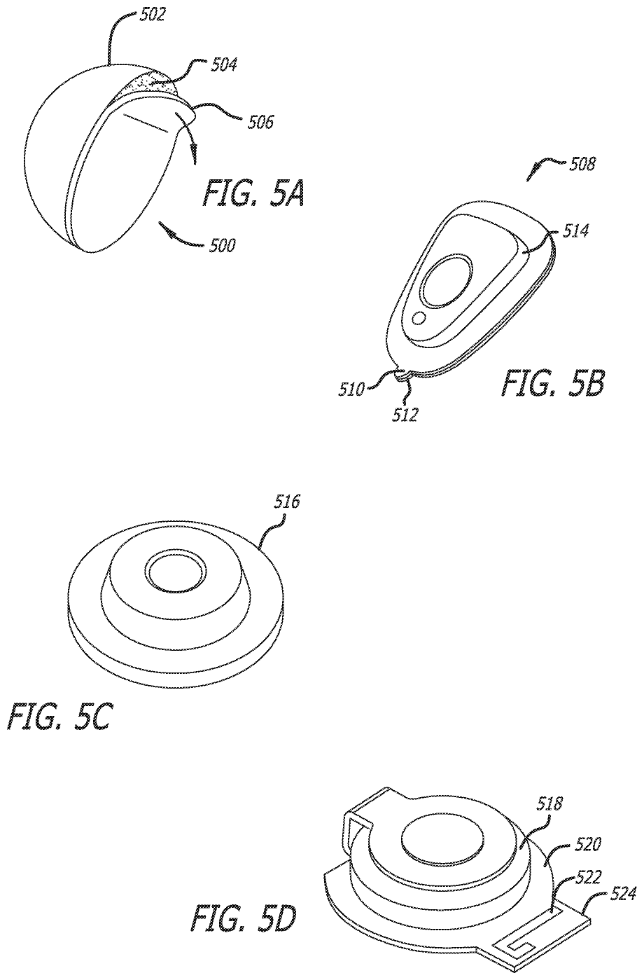

Referring to FIGS. 5A-5D, example monitoring devices of the TTM/neurostimulation system of FIG. 1 are shown according to some embodiments. By way of example, and with reference to FIG. 4, a motion sensor 500 is shown that includes an accelerometer housed within a housing 502 having an adhesive backing 504 and removable liner 506 initially provided therewith. To initiate patient use, the liner 506 may be selectively removed, wherein the adhesive backing 504 may be mounted to a jaw of a patient P. In one approach, an on-board battery may be housed in housing 502, e.g. for powering the accelerometer and an on-board transmitter for transmitting a monitoring signal 214 indicative of a magnitude of motion of the patient's chin. The motion sensor 502 may transmit a monitoring signal to the transceiver 210 control module 102 that is indicative of a degree of motion of the patient P's chin. More particularly, the motion sensor 500 may comprise a transceiver and rectifier arrangement for receiving a query/power signal from the control module 102, transducing electrical energy therefrom, and using the energy to generate and transmit the monitoring signal. As may be appreciated, a plurality of motion sensors 500 may be employed. The monitoring signal(s) may be processed at the control module 102 in accordance with the described functionalities to provide an output (e.g. a visual or auditory output) at a user interface 206, or otherwise. As previously noted, the output may provide an indication of a magnitude or stage of patient shivering. Additionally or alternatively, such output may provide information relating to available neurostimulation procedures, e.g., based on the neurostimulation device 110.

Reference is now made to FIGS. 5B, 5C and 5D illustrating other embodiments of a motion sensor. As shown in FIG. 5B, the sensor 508 may include a base pad 510 initially provided with a removable liner 512 overlaying an adhesive bottom surface of the base pad 510. As may be appreciated, the liner 512 may be selectively removed prior to adhesive interconnection of the motion sensor 508 to the patient P. The motion sensor 508 further includes a housing portion 514 that houses a sealed sensor assembly 516, which is shown in FIG. 5C. As illustrated in FIG. 5D, of the sensor assembly 516 may include an accelerometer module 518 that is located between opposing circuit elements mounted on opposing, inside surface(s) of a wrap-around circuit board 520. In the illustrated embodiment, a transceiver device 416, e.g. an RF antenna, may be patterned on a stub portion 524 of the circuit board 414 for wireless transmission/reception of monitoring signals and power signals. In the later regard, circuit correspondingly located on circuit board 520 may include a rectifier and/or battery for powering the sensor operations. In other embodiments, the patterned antenna 522 may be replaced by a chip transceiver mounted on the circuit board 520.

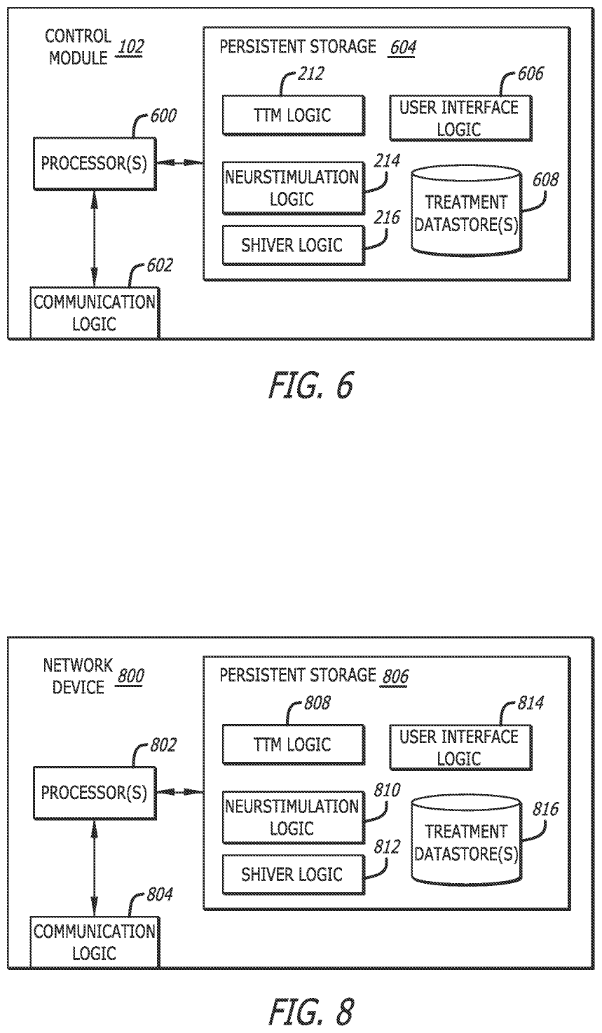

Referring to FIG. 6, a logical representation of the control module of FIG. 2 is shown according to some embodiments. The control module 102 comprises one or more processors 600 that are coupled to the communication interface 602, which enables communication with other network devices, such as a clinician's mobile device, a neurostimulation device, and/or any of the sensors discussed in the disclosure. According to one embodiment of the disclosure, the communication interface 602 may be implemented as a physical interface including one or more ports for wired connectors. Additionally, or in the alternative, communication interface 602 may be implemented with one or more radio units for supporting wireless communications with other network devices.

The processor(s) 600 is further coupled to the persistent storage 604, e.g., non-transitory, computer-readable medium. According to one embodiment of the disclosure, the persistent storage 604 may include (i) the TTM logic 212, (ii) the neurostimulation logic 214, (iii) the shiver logic 216, (iv) the user interface logic 606, and (v) one or more treatment database(s) 608. Of course, when implemented as hardware, one or more of these logic units could be implemented separately from each other. As referenced above, the TTM logic 212 includes logic, algorithms and/or data that, upon execution by the processor 208, cause performance of operations that instruct the heat exchange system 200 to warm or cool fluid circulating through the pads 104. Further, the neurostimulation logic 214 includes logic, algorithms and/or data that, upon execution by the processor 208, cause performance of operations that instruct the neurostimulation device 110 to initiate, alter or terminate a neurostimulation procedure on the patient P. The shiver logic 216 includes logic, algorithms and/or data that, upon execution by the processor 208, cause performance of operations that analyze the signals received from at least the monitoring device 108 to determine a level of shiver of the patient P.

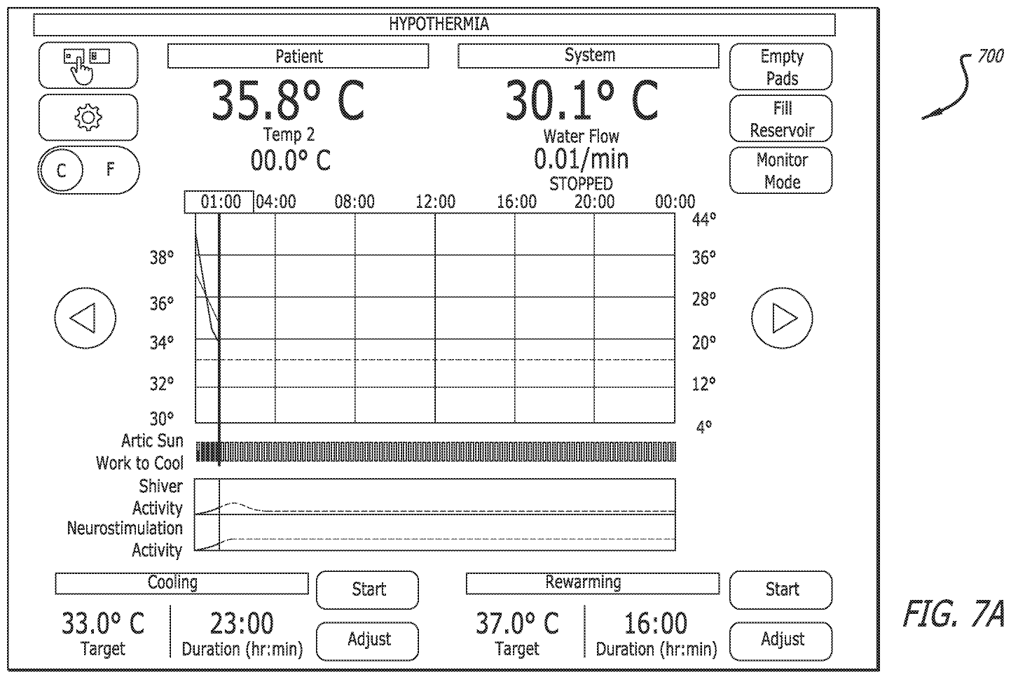

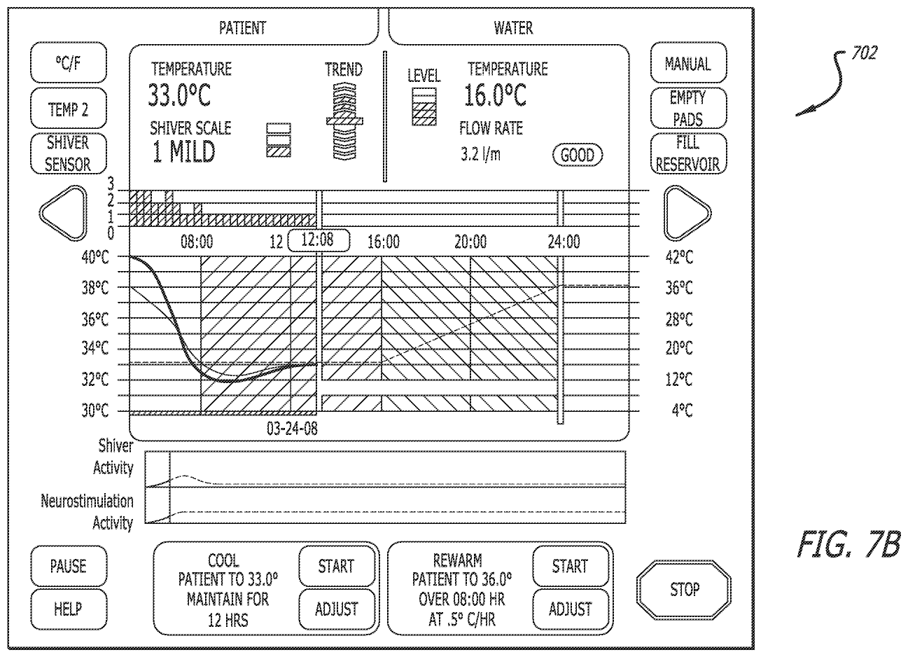

Referring to FIGS. 7A-7B, example user interface displays employable in conjunction with implementations of the TTM/neurostimulation system of FIG. 1 are shown according to some embodiments. As illustrated, various user interfaces may be generated and provided by the control module 102 that are configured to receive user input indicating information related to the circulation of cooled and/or warmed fluid through pads 104 to adjust a patient's temperature in accordance with a predetermined and/or otherwise controllable protocol. Additionally, various user interfaces display information relating to an amount of shiver by the patient P, such as a magnitude or a relative amount displayed via a scale. Yet other user interfaces may provide information relevant to a neurostimulation procedure. It should be understood that various user interfaces may be configured and designed to display any of the information discussed in the disclosure on a single user interfaces or on separate user interfaces.

As shown in FIG. 7A, an interactive screen 700 may be provided at user interface 206 which includes a graphic display portion that graphically illustrates temperature-related data in a first region, e.g., as a function of time, and that further illustrates patient motion data, e.g. shivering data, as a function of time in a second region. The first region may present a first plot of a target patient temperature level as a function of time, e.g. a predetermined patient temperature adjustment rate plot reflecting a desired patient temperature to be reached by controlling the temperature of the circulated fluid. Further, a second plot of a measured patient temperature as a function of time may be presented. Additionally, a third plot of a measured temperature of the fluid circulated by the control module 102 though the pads 104 may be provided.

FIG. 7B provides an alternative example of the information of FIG. 7A displayed in a varied form. For example, as shown by FIG. 7B, a region of an interactive screen 702 may be provided to visually display patient motion data in relation to a predetermined magnitude scale, e.g., a “shiver scale.” By way of example, a plurality of predetermined levels of patient motion, or degrees of shivering, may be graphically presented as a function of time. In the illustrated example, four levels of detected patient motion may be provided to a user, wherein no visual indication is provided for a low, or “zero” level of motion, and wherein increasing level of motions may be graphically presented by one, two or three stacked “box” indicators.

As may be appreciated, by visually monitoring the magnitude of shivering response displayed on the screen 702, medical personnel may assess the need and/or desirability for taking responsive action. For example, such responsive action may include the initiation of a neurostimulation procedure and/or a modification to the patient cooling/warming protocol discussed hereinabove (e.g. decreasing a target patient cooling rate and/or an increasing targeted temperature for patient cooling). As discussed herein, the logic of the control module 102 may analyze received signals and automatically initiate a neurostimulation procedure, for example, when the signals received from the monitoring device 108 indicate the patient P shiver is above a predetermined threshold. It should be understood that FIGS. 7A-7B provide examples of the multiple of ways by which the information provided in the disclosure may be illustrated on a user interface.

Referring to FIG. 8, a logical representation of logic configured to control aspects of the TTM/neurostimulation system of FIG. 1 deployed on an application on network device 800 is shown according to some embodiments. The network device 800 comprises one or more processors 802 that are coupled to the communication interface 804, which enables communication with other network devices, such as the control module 102. According to one embodiment of the disclosure, the communication interface 804 may be implemented as a physical interface including one or more ports for wired connectors. Additionally, or in the alternative, communication interface 804 may be implemented with one or more radio units for supporting wireless communications with other network devices.

The processor(s) 802 is further coupled to the persistent storage 806, e.g., non-transitory, computer-readable medium. According to one embodiment of the disclosure, the persistent storage 806 may include (i) the TTM logic 808, (ii) the neurostimulation logic 810, (iii) the shiver logic 812, (iv) the user interface logic 814, and (v) one or more treatment database(s) 816. Of course, when implemented as hardware, one or more of these logic units could be implemented separately from each other. Each of the TTM logic 808, the neurostimulation logic 810, and the shiver logic 812 may include all or a portion of the corresponding logic described above with respect to FIG. 2. For instance, each of the TTM logic 808, the neurostimulation logic 810, and the shiver logic 812 may include logic that communicates with the corresponding logic stored on the control module 102 (e.g., the TTM logic 808 corresponds to the TTM logic 212) such that the logic stored on the network device 800 may initiate any procedure of the corresponding logic and/or receive the same signals received by the corresponding logic of the control module 102. In other embodiments, the logic stored on the network device 800 may merely receive signals from the control module 102 (e.g., potentially mirroring those received by the logic of the control module 102) and enable display of such information as a result of processing of the user interface logic 612. In order to initiate a procedure, the logic of the network device 800 may query the one or more treatment database 816, which may store preset procedures, protocols or other information relevant to initiating a TTM therapy and/or a neurostimulation procedure.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A patient temperature control system, comprising:
a control module configured to provide fluid that is heated or cooled, wherein the control module includes a circulating pump;
a heating and cooling system configured to couple with the control module and receive the fluid, wherein circulating pump is configured to circulating the fluid through the heating and cooling system;
a temperature sensor coupled to the control module, the temperature sensor configured to measure a body temperature of a patient and provide signals to the control module that indicate the body temperature; and
a neurostimulation device coupled to the control module and configured to provide neurostimulation to the patient,
wherein the control module includes logic, stored on non-transitory, computer-readable medium that, when executed by one or more processors, causes performance of operations including generation and transmission of first instructions to the neurostimulation device causing initiation of a first neurostimulation procedure.

2. The system of claim 1, wherein the control module includes a heat exchange system for heating or cooling the fluid.

3. The system of claim 1, wherein the logic, when executed by the one or more processors, causes performance of further operations including:
receiving the signals indicating the body temperature;
analyzing the body temperature to determine whether a temperature of the fluid is to be adjusted; and
providing second instructions to the heat exchange system indicating whether the fluid is to be heated, cooled, or maintained at a current temperature.

4. The system of claim 1, wherein the control module further includes a fluid reservoir that is maintained at substantially atmospheric pressure.

5. The system of claim 1, further comprising a motion sensor device configured to measure an amount of motion of the patient indicating a level of shiver.

6. The system of claim 1, wherein the heating and cooling system includes one or more contact pads configured to directly or indirect contact the patient and receive the fluid.

7. The system of claim 1, wherein the neurostimulation device includes an external electrode and an internal electrode, and is configured to provide deep brain stimulation.

8. The system of claim 1, wherein the neurostimulation device includes an external electrode and an internal electrode placed within the patient's skull, and is configured to provide deep brain stimulation.

9. The system of claim 1, wherein the neurostimulation device includes at least a first set of electrodes for placement on the patient's skill, the first set of electrodes including an emitter and a receiver each configured to receive power via a power supply device, wherein the neurostimulation device is configured to provide noninvasive brain stimulation.

10. The system of claim 1, wherein the neurostimulation device is a wrist-worn device.

11. The system of claim 1, wherein the neurostimulation is configured to target the hypothalamus to control thermoregulation of the patient's body and prevent or reduce patient shiver.

12. A method of providing targeted temperature management and neurostimulation, the method comprising
providing a targeted temperature management (TTM) procedure to a patient by circulating cooled fluid through a heating and cooling system directly or indirectly in contact with the patient;
obtaining a measurement indicating an amount of shiver by a body of the patient;
determining, based at least in part on the measurement indicating the amount of shiver, a neurostimulation procedure to be initiated on the patient; and
providing the neurostimulation procedure to the patient.

13. The method of claim 12, wherein the TTM procedure is provided by a combination of a control module configured to provide the cooled fluid and a circulating pump configured to circulate the cooled fluid through the heating and cooling system.

14. The method of claim 13, wherein the control module receives a measurement of a body temperature of the patient from a temperature sensor.

15. The method of claim 13, wherein the control module includes logic, stored on non-transitory, computer-readable medium that, when executed by one or more processors, causes performance of operations including generation and transmission of first instructions to a neurostimulation device causing the providing of the neurostimulation procedure to the patient.

16. The method of claim 12, wherein the neurostimulation device includes an external electrode and an internal electrode placed within the patient's skull, and is configured to provide deep brain stimulation.

17. The method of claim 12, wherein the neurostimulation device includes at least a first set of electrodes for placement on the patient's skill, the first set of electrodes including an emitter and a receiver each configured to receive power via a power supply device, wherein the neurostimulation device is configured to provide noninvasive brain stimulation.

18. The method of claim 12, wherein the neurostimulation device is a wrist-worn device.

19. The method of claim 12, wherein the neurostimulation procedure is configured to target the hypothalamus to control thermoregulation of the patient's body and prevent or reduce patient shiver.

20. The method of claim 12, wherein the control module further includes a fluid reservoir that is maintained at substantially atmospheric pressure.
